SEQUENCE LISTING (6 sequences)

SEQ ID NO: 1 - DNA, 64 bp, Artificial sequence
  Synthetic oligonucleotide used for cloning the CKI-alpha specific shRNA expression cassette
gatccccaagaagatgtccacgcctgttcaagagacaggcgtggacatcttctttttttggaaa

SEQ ID NO: 2 - DNA, 64 bp, Artificial sequence
  Synthetic oligonucleotide used for cloning the CKI-alpha specific shRNA expression cassette
agcttttccaaaaaaagaagatgtccacgcctgtctcttgaacaggcgtggacatcttcttggg

SEQ ID NO: 3 - DNA, 63 bp, Artificial sequence
  Synthetic oligonucleotide used for cloning the CKI-delta/epsilon specific shRNA expression cassette
gatcccgggcttctcctatgactacttcaagagagtagtcataggagaagccctttttggaaa

SEQ ID NO: 4 - DNA, 64 bp, Artificial sequence
  Synthetic oligonucleotide used for cloning the CKI-delta/epsilon specific shRNA expression cassette
agcttttccaaaaagggcttctcctatgactactctcttgaagtagtcataggagaagcccggg

SEQ ID NO: 5 - DNA, 57 bp, Artificial sequence
  Synthetic oligonucleotide used for cloning the CKI-delta specific shRNA expression cassette
ccggcccatcgaagtgttgtgtaaactcgagtttacacaacacttcgatgggttttt

SEQ ID NO: 6 - DNA, 21 bp, Artificial sequence
  An exemplary siRNA capable of down-regulating CKI-delta
gaaacaugguguccgguuutt

(19) United States
(12) Patent Application Publication
(10) Pub. No.: US 2019/0365754 A1
(43) Pub. Date: Dec. 5, 2019

(54) METHOD OF ELIMINATING STEM CELLS

(71) Applicant: Yissum Research Development Company Of The Hebrew University Of Jerusalem Ltd., Jerusalem (IL)

(72) Inventors: Yinon Ben-Neriah, Mevasseret Zion (IL); Waleed Minzel, Krar-Kana (IL); Guy Brachya, Jerusalem (IL)

(21) Appl. No.: 16/228,630

(22) Filed: Dec. 20, 2018

Related U.S. Application Data
(63) Continuation of application No. 15/115,712, filed on Aug. 1, 2016, now abandoned.
(60) Provisional application No. 61/934,954, filed on Feb. 3, 2014.

(51) Int. Cl. A61K 31/506; C12N 15/113; A61K 35/28; A61K 35/14

(52) U.S. Cl. CPC A61K 31/506; C12N 15/1137; C12N 2310/14; A61K 35/14; A61K 35/28

(57) ABSTRACT

A method of treating cancer in a subject is disclosed. The method comprises administering to the subject a therapeutically effective amount of a Casein kinase I alpha (CKlalpha) inhibitor, wherein the cancer is not associated with an Adenomatous polyposis coli (APC) mutation. Additional uses of CKI inhibitors are also disclosed.

### FIELD AND BACKGROUND OF THE INVENTION

The present invention, in some embodiments thereof, relates to methods of eliminating stem cells including hematopoietic stem cells and cancer stem cells.

The Wnt pathway is highly conserved throughout evolution, from worms to man, playing crucial roles in embryonic development and diseases. Wnt signaling is strictly regulated by a set of kinases and phosphatases, acting on different components of the cascade and leading to various cell fates during an organism's life.

The main target of the canonical Wnt pathway is cytoplasmic β-catenin, which serves as a transcription co-activator for genes of proliferation, differentiation, migration and survival. The transduction of signal depends on the presence or absence of the Wnt ligand. In resting tissues, in the absence of Wnt ligand, β-catenin is constantly phosphorylated and degraded by a multiprotein complex, and is thus maintained at low levels in cells. In dividing cells, in adult's self-renewing tissues and throughout embryogenesis, secreted Wnt proteins bind to members of the Frizzled receptor family and to the coreceptor LRP5/6 on the cell membrane. Wnt binding activates Dishevelled (Dv1), resulting in dissociation of β-catenin degradation complex and stabilization of β-catenin in the cytoplasm. This enables the translocation of β-catenin into the nucleus and the activation of its target genes (e.g. c-Myc, cyclin D1) through Tcf/Lef-dependent transcription. Deregulation of the canonical Wnt signal leads to various cancers, among which is colorectal carcinoma (CRC), hepatocellular carcinoma (HCC) and melanoma. In such cancers, one or more Wnt component is often mutated, resulting in aberrant accumulation of nuclear β-catenin. This explains the requirement for tight regulation on β-catenin levels in the cell.

The mechanism by which β-catenin is phosphorylated and degraded has been revealed only recently, emphasizing significant players in the Wnt signaling pathway. The β-catenin degradation complex consists of the Adenomatous polyposis coli (APC) tumor suppressor, Axin1 or Axin2 (which are thought to play a scaffold function), and of two Serine/Threonine kinases: Casein kinase I (CKI) and Glycogen synthase kinase-3 (GSK3), which phosphorylate β-catenin on four N-terminal Ser/Thr residues. This event marks β-catenin for ubiquitination by the SCF^{β-TrcP} E3 ubiquitin ligase and subsequent proteasomal degradation. It has been shown lately that the first phosphorylation event is mediated by CKI, which phosphorylates Ser45 of β-catenin. This creates a priming site for GSK3, which subsequently phosphorylates Thr41, Ser37 and Ser33. The last two residues, when phosphorylated, serve as a docking site for the E3 ligase βTrCP, which marks β-catenin for degradation.

CKI's involvement was proven to be both necessary and sufficient for driving the cascade leading to β-catenin down-regulation. This is in agreement with studies on Wnt components' homologues in Drosophila and therefore assigns CKI as a Wnt antagonist. On the other hand, developmental studies in Xenopus and C. elegans implicated CKI as a Wnt effector, showing that CKI promotes secondary body axis and embryonic polarity (Wnt effects). Supporting that is the observation that CKI phosphorylates and activates Dv1, another Wnt effector, thereby increasing β-catenin levels.

U.S. Patent Application No. 20050171005 teaches methods of modulating β-catenin phosphorylation.

U.S. Patent Application No. 20090005335 teaches treating cancer cells which have a mutation in the APC gene by providing compositions which up-regulate B-catenin.

U.S. Patent Application No 20110076683 teaches Wnt inhibitors for the treatment of leukemias.

Additional background art includes U.S. Patent Application No. 20080146555, WO 2014023271 and U.S. Patent Application No. 20100179154.

### SUMMARY OF THE INVENTION

According to an aspect of some embodiments of the present invention there is provided a method of treating a cancer in a subject in need thereof, comprising administering to the subject a therapeutically effective amount of a Casein kinase I alpha (CKIα) inhibitor, wherein the cancer is not associated with an Adenomatous polyposis coli (APC) mutation, thereby treating the cancer.

According to an aspect of some embodiments of the present invention there is provided a use of a Casein kinase I alpha (CKIα) inhibitor for treating cancer, wherein the cancer is not associated with an Adenomatous polyposis coli (APC) mutation.

According to an aspect of some embodiments of the present invention there is provided a method of treating cancer in a subject in need thereof comprising administering to the subject a therapeutically effective amount of PF670462, wherein the cancer is not chronic lymphocytic leukemia (CLL), thereby treating the cancer.

According to an aspect of some embodiments of the present invention there is provided a use of PF670462 for treating cancer, wherein the cancer is not CLL.

According to an aspect of some embodiments of the present invention there is provided a method of treating chronic myelogenous leukemia (CML) in a subject in need thereof, comprising administering to the subject a therapeutically effective amount of a Casein kinase I inhibitor, wherein the CML is selected from the group consisting of imatinib-resistant CML, imatinib-related TKI-resistant CML, imatinib-intolerant CML, accelerated CML, and lymphoid blast phase CML, thereby treating the CML.

According to an aspect of some embodiments of the present invention there is provided a use of Casein kinase I inhibitor for treating CML, wherein the CML is selected from the group consisting of imatinib-resistant CML, imatinib-intolerant CML, accelerated CML, and lymphoid blast phase CML.

According to an aspect of some embodiments of the present invention there is provided a method of transplanting cells into a subject in need thereof comprising:

(a) depleting immature blood cells from a blood or bone marrow of a subject by contacting the immature blood cells from a blood or bone marrow with an amount of a CKI inhibitor which up-regulates an amount and/or activity of p53 and kills the immature blood cells in the blood or bone marrow; and subsequently

(b) transplanting cells into the subject.

According to an aspect of some embodiments of the present invention there is provided a method of depleting immature blood cells from a blood or bone marrow of a subject comprising contacting the stem cells ex vivo with an amount of a CKI inhibitor which up-regulates an amount and/or activity of p53 and kills the immature blood cells in the blood or bone marrow, thereby depleting the immature blood cells from the blood or bone marrow.

According to an aspect of some embodiments of the present invention there is provided a method of identifying and optionally producing an agent useful for depleting stem cells the method comprising:

(a) determining an activity and/or expression of CKI in a presence of the candidate agent;

(b) selecting the agent which down-regulates an activity and/or expression of the CKI and upregulates an activity and/or expression of p53, thereby identifying an agent useful for eliminating stem cells.

According to an aspect of some embodiments of the present invention there is provided a composition of matter comprising a small molecule which has at least a two fold greater inhibitory activity towards CKI alpha than towards CKI delta and/or CKI epsilon.

According to some embodiments of the invention, the method further comprises inducing mobilization of the immature blood cells from the bone marrow to the blood prior to the depleting.

According to some embodiments of the invention, the CKlalpha inhibitor is at least as effective in upregulating p53 as an inhibitor of CKI delta and epsilon.

According to some embodiments of the invention, the inhibitor binds to CKIα or a polynucleotide encoding same.

According to some embodiments of the invention, the inhibitor binds to CKI or a polynucleotide encoding same.

According to some embodiments of the invention, the inhibitor activates a DNA damage response (DDR).

According to some embodiments of the invention, the CKI inhibitor comprises a CKIα inhibitory activity.

According to some embodiments of the invention, the CKI inhibitor further comprises a CKI delta and/or CKI-epsilon inhibitory activity.

According to some embodiments of the invention, the CKI inhibitor comprises a CKI delta and CKI-epsilon inhibitory activity.

According to some embodiments of the invention, the inhibitor is a small molecule inhibitor.

According to some embodiments of the invention, the inhibitor is PF670462.

According to some embodiments of the invention, the inhibitor is an RNA silencing agent.

According to some embodiments of the invention, the silencing agent is targeted against CKIα.

According to some embodiments of the invention, the immature blood cells comprise stem cells.

According to some embodiments of the invention, the immature blood cells comprise cancer stem cells.

According to some embodiments of the invention, the contacting is effected in vivo.

According to some embodiments of the invention, the contacting is effected ex vivo.

According to some embodiments of the invention, the contacting is effected during apheresis.

According to some embodiments of the invention, the depleting is effected without irradiation or chemotherapy.

According to some embodiments of the invention, the depleting is effected in combination with irradiation and/or chemotherapy.

According to some embodiments of the invention, the cancer is a hematological malignancy.

According to some embodiments of the invention, the hematological malignancy is selected from the group consisting of Chronic Myelogenous Leukemia (CML), CML accelerated phase, or blast crisis, multiple myeloma, Hypereosinophilic Syndrome (HES), myelodysplastic syndrome (MDS), acute lymphocytic leukemia (ALL), acute myeloid leukemia (AML), acute promyelocytic leukemia (APL), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myeloproliferative disorders (MPD), multiple myeloma, (MM) and myeloid sarcoma.

According to some embodiments of the invention, the hematological malignancy is Chronic Myelogenous Leukemia (CML).

According to some embodiments of the invention, the CML is selected from the group consisting of imatinib-resistant CML, imatinib-intolerant CML, imatinib-related TKI-resistant CML, accelerated CML, and myeloid or lymphoid blast phase CML.

According to some embodiments of the invention, the method further comprises administering to the subject Imatinib.

According to some embodiments of the invention, the subject is not administered with an agent selected from the group consisting of Imatinib, Dastinib and Nilotinib.

According to some embodiments of the invention, the cancer is breast cancer or melanoma.

According to some embodiments of the invention, the CKIα inhibitor has at least twice the inhibitory activity for CKIα than CKldelta or CKlepsilon.

According to some embodiments of the invention, the stem cells comprise hematopoietic stem cells (HSCs).

According to some embodiments of the invention, the stem cells comprise cancer stem cells.

According to some embodiments of the invention, the method further comprises testing an effect of the candidate agent as a treatment for cancer or as a pre-treatment prior to cell transplantation.

According to some embodiments of the invention, the method further comprises synthesizing the candidate agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F illustrate that CKIα ablation depletes mice of hematopoietic stem cells (HSC) and allows bone marrow engraftment.

A) Scheme of generating a chimera mice. B) Absolute counts of LT-HSC (Lineage⁻c-kit⁺Sca1⁺CD34⁻FLT3⁻), ST-HSC (Lineage⁻c-kit⁺Sca1⁺CD34⁺FLT3⁻), MPP (Lineage⁻c-kit⁺Sca1⁺CD34⁺FLT3⁺) from two femurs and two tibias at day 7 post induction. C) Survival curve of lethally IR mice engrafted with either CKIα KO bone marrow or control mice. D) Scheme of CKIα reconstitution experiment. E) Survival curve of CKIα KO induced mice reconstituted or not treated. F) Percentage of GFP+ Peripheral blood leukocytes from either WT or CKIα KO induced mice 3 months after reconstitution.

FIG. 2 is a scheme of generation of mouse model of CML blast crisis.

FIGS. 3A-D illustrates how CKIα ablation prevents CML development. A) Experimental scheme. B) Percentage of GFP+ peripheral blood leukocytes monitored for two months following transplantation of leukemia initiating cells (LICs); mice with no CKIα deletion (PBS-treated or LICs carrying no MXCre were moribund within 3 weeks and were sacrificed. C) Photographs of blood smears from leukemic mice where CKIα deletion is induced (pIpC-treated) or not induced (PBS treated). D) Survival of leukemic mice where CKIα deletion is either induced with pIpC, or not induced (PBS-treated), or having no MXCre for deletion).

FIGS. 4A-C illustrates how CKIα ablation depletes both normal and leukemic stem cells and allows normal bone marrow reconstitution. A) Experimental scheme. B) Percentage of CD45.1 (donor cells) and GFP+ leukemic cells among peripheral blood leukocytes 12 days following leukemia-afflicted BM transplantation, showing normal donor BM reconstitution (CD45.1) only after CKIα deletion (red bars) and high percentage of leukemic GFP-positive cells without CKIα deletion (PBS treatment, black bars). C) Full survival of leukemic mice following CKIα deletion (upon pIpC treatment), due to successful donor marrow reconstitution.

FIGS. 5A-D illustrates that the CKI inhibitor PF670462 preferentially targets the leukemia cells in vitro. A) Experimental scheme. B) RT-PCR results illustrating dose-dependent increase in expression of p53 and Wnt targets upon inhibitor treatment. C) Leukemic cell number is selectively reduced following PF670462 treatment—dose response curve with LD50<204. D) apoptotic gene expression is increased in leukemic cells following PF670462 treatment.

FIGS. 6A-F illustrate that PF670462 activates both Wnt and p53 in the BM, eliminates the transplanted leukemia-initiating cells, and prevents CML development in vivo. A) Experimental treatment scheme. B) Western blot analysis illustrating β-catenin and p53 stabilization as well as elevation of c-Myc (an example of a Wnt target gene) upon PF670462 treatment. C) Percentage of GFP+ peripheral blood leukocytes following PF670462 treatment shows rapid expansion of GFP+ leukemia cells in the peripheral blood of vehicle-treated mice and no expansion in inhibitor-treated mice. D) H&E staining of bone marrow vertebrate sections showing blast cell invasion and complete destruction of the vertebrate in vehicle-treated mice and a normal vertebrate in inhibitor-treated mice. Moribund vehicle-treated mice were sacrificed 12 days after leukemia transplant. Healthy inhibitor-treated mice were sacrificed after 3 weeks and their bone marrow transplanted to irradiate mice to monitor both normal long term hematopoiesis and no leukemia relapse. E) Immature myeloid cells and blasts in vehicle-treated mice two days before succumbing and normal peripheral blood picture in inhibitor-treated mice at the same time. F) Survival of leukemic mice following PF670462 treatment.

FIGS. 7A-B are photographs illustrating the effect of CKI alpha deletion in a melanoma mouse model. FIG. 7A are photographs depicting the ear of a BrafV600E; Pten-double floxed mouse face and histology, prior to and 56 days following local ear tamoxifen administration. FIG. 7B are photographs depicting the ear of a BrafV600E; Pten; CKIα-triple floxed mouse, prior to (left) and 56 d following (right) local ear tamoxifen induction. No tumors are visible following tamoxifen induction in B, only ear pigmentation, attesting to a strong tumor suppressor effect of CKIα deletion, with no tumor mutant escape.

### DESCRIPTION OF SPECIFIC EMBODIMENTS OF THE INVENTION

The present invention, in some embodiments thereof, relates to methods of eliminating stem cells including hematopoietic stem cells and cancer stem cells.

The principles and operation of the method of eliminating stem cells according to the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The β-catenin degradation complex consists of the Adenomatous polyposis coli (APC) tumor suppressor, Axin1 or Axin2 (which are thought to play a scaffold function), and of two Serine/Threonine kinases: Casein kinase I (CKI) and Glycogen synthase kinase-3 (GSK3), which phosphorylate β-catenin on four N-terminal Ser/Thr residues. Both CKIα and APC are noted to play a role in Wnt signaling and mitotic spindle regulation.

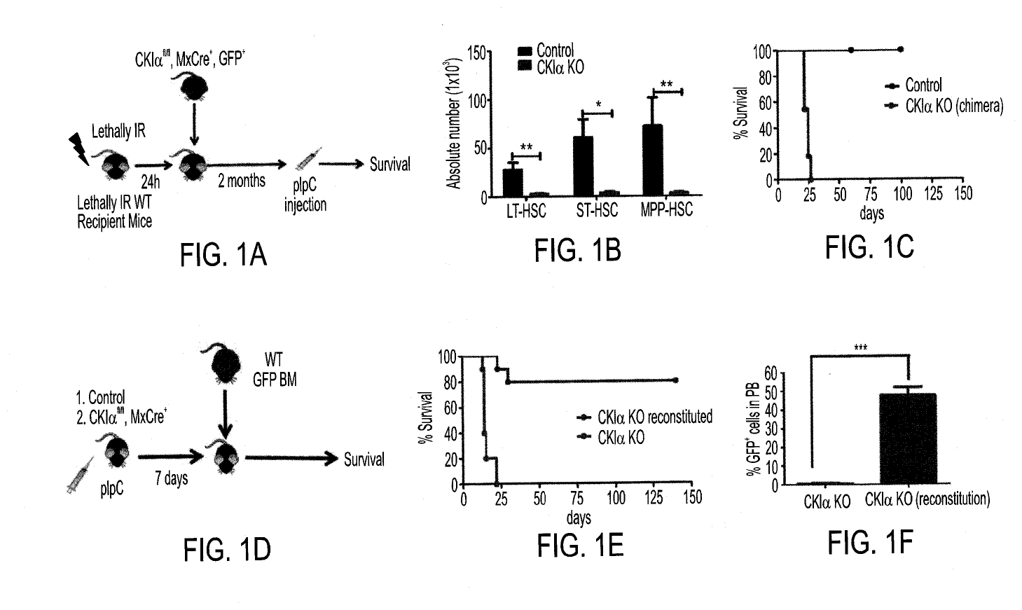

In order to analyze the role played by CKI in bone marrow stem cells such as hematopoietic stem cells (HSCs), the present inventors generated conditional bone marrow CKIα knock-out mutant mice. As illustrated in FIGS. 1A-F, bone marrow CKIα ablation depletes mice of hematopoietic stem cells (HSC) and allows bone marrow engraftment (FIGS. 1A-F) with no further means commonly used for transplantation preconditioning (e.g., irradiation or chemotherapy).

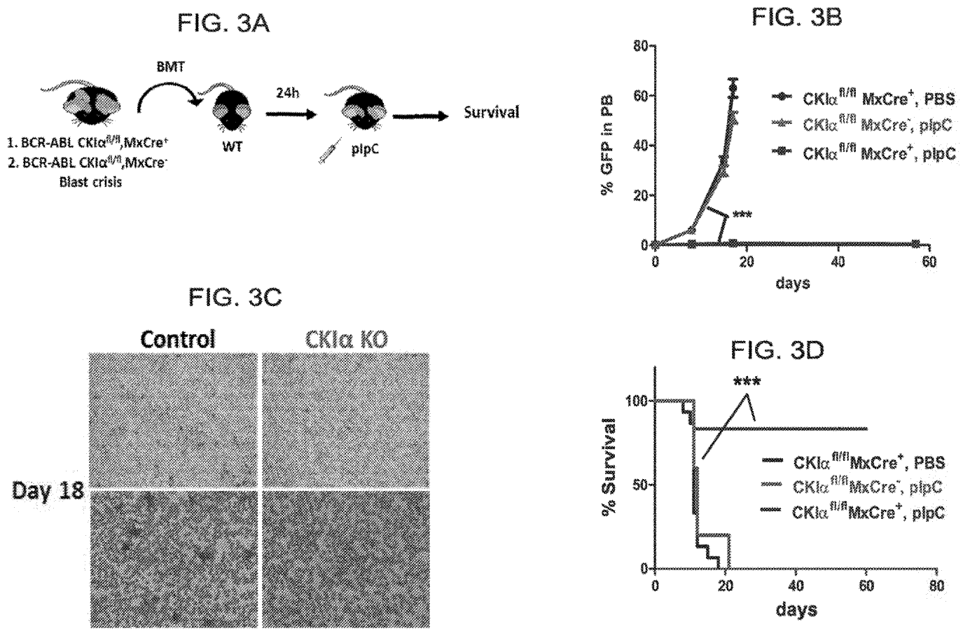

Using a mouse model of CML blast crisis, the present inventors went on to show that CKIα ablation prevents Chronic Myelogenous Leukemia (CML) development (FIGS. 3A-D). Since CML in general, and the blast crisis stage in particular, is known to be associated with cancer stem cells, the present inventors surmise that CKIα inhibitors may be used to deplete not only hematopoietic stem cells (HSC), but other stem cells such as cancer stem cells.

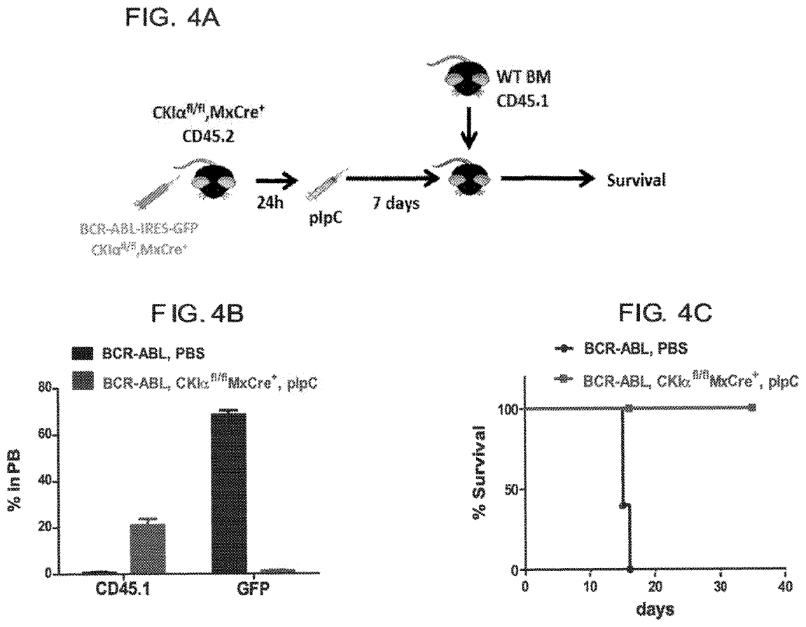

To evaluate if CKIα deletion can substitute chemotherapy or irradiation-induces myeloablation and leukemia cell clearance, leukemic cells were injected into CKIα floxed with Mx-Cre mice. When knock-out was not induced, a very high percentage of leukemic cells were present, while in the CKIα KO, the leukemic cells were undetectable (FIG. 4B), as mirrored by the survival rate data (FIG. 4C).

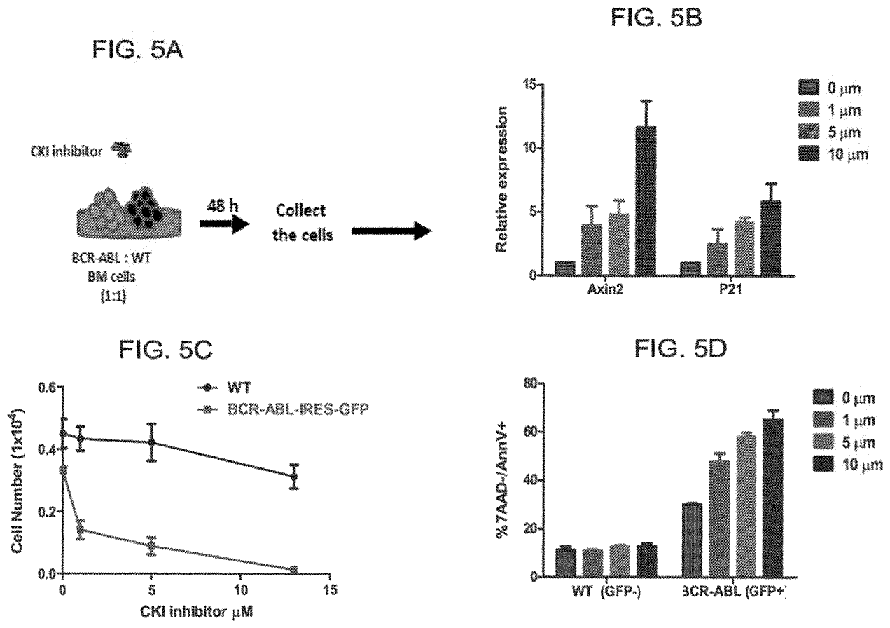

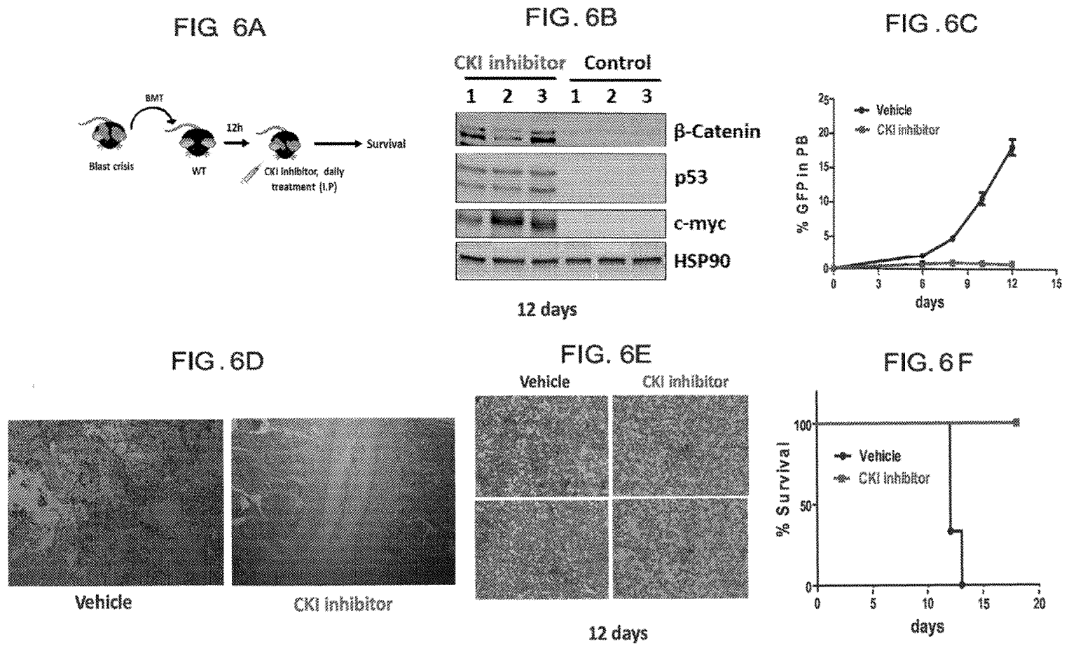

The present inventors sought to confirm their results using a small molecule agent which inhibits CKI. Since downregulation of CKIα is known to increase the expression of p53, the present inventors searched for CKI inhibitors which had a similar effect on p53. It can be seen in FIGS. 5B and 6B that the CKI inhibitor PF670462 substantially increased the expression of p53 and its targets in bone marrow cells. In an in vitro study, the present inventors showed that PF670462 preferentially depletes leukemic cells. The profound effect of this inhibitor was mirrored in an in vivo study. Thus, PF670462 was shown to eliminate transplanted leukemia-initiating cells, and prevents CML development in vivo (FIGS. 6C-F). No leukemia cells were evident upon transplantation of the bone marrow of inhibitor-treated mice to lethally irradiated mice (one month following transplantation), indicating that the inhibitor treatment eradicated the leukemia stem cells, while preserving the normal hematopoietic stem cells.

Whilst further reducing the present invention to practice, the present inventors analyzed the effect of CKIα depletion on additional cancers and found that CKIα knockout had a therapeutic effect in a mouse model for melanoma.

Since melanoma is derived from cells of the neuroectoderm germ layer and leukemic cells are derived from cells of the mesoderm germ layer, the present inventors deduce that downregulation of CKI can be effective for a myriad of cancers, irrespective of the germ layer from which the tumor cells are derived. Furthermore, since CKI inhibition has been shown to selectively target cancer stem cells, the present inventors conclude that agents capable of CKI inhibition should be effective against cancer stem cells in general, irrespective of the particular cancer in which they are involved.

Thus, according to a first aspect of the present invention there is provided a method of treating a cancer in a subject in need thereof, comprising administering to the subject a therapeutically effective amount of a Casein kinase Ia inhibitor, wherein the cancer is not associated with an Adenomatous polyposis coli (APC) mutation, thereby treating the cancer.

The term “cancer” as used herein refers to proliferative diseases including but not limited to carcinoma, lymphoma, blastoma, sarcoma, and leukemia. The cancer may for example be a solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

According to a particular embodiment, the cancer is a melanoma, a breast cancer or a hematological malignancy.

The term “hematological malignancy” herein includes a lymphoma, leukemia, myeloma or a lymphoid malignancy, as well as a cancer of the spleen and the lymph nodes. Exemplary lymphomas that are amenable to treatment with the disclosed anti-CXCR4 antibodies of this invention include both B cell lymphomas and T cell lymphomas. B-cell lymphomas include both Hodgkin's lymphomas and most non-Hodgkins lymphomas. Non-limiting examples of B cell lymphomas include diffuse large B-cell lymphoma (DLBCL), follicular lymphoma (FL), mucosa-associated lymphatic tissue lymphoma (MALT), small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma (NMZL), splenic marginal zone lymphoma (SMZL), intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, acute myelogenous leukemia (AML; also called acute lymphoid leukemia), chronic myelogenous leukemia (CML), B-cell prolymphocytic leukemia (B-PLL), acute lymphoblastic leukemia (ALL) and myelodysplasia (MDS). Hematological malignancies further include myelomas, such as, but not limited to, multiple myeloma (MM), smoldering multiple myeloma (SMM) and B-cell chronic lymphocytic leukemia (CLL).

According to a particular embodiment, the hematological malignancy is chronic myelogenous leukemia (CML). The term CML includes imatinib-resistant CML, CML tolerant to second/third generation Bcr-Abl TKIs (e.g, dasatinib and nilotinib), imatinib-intolerant CML, accelerated CML, and lymphoid blast phase CML.

Other hematological and/or B cell- or T-cell-associated cancers are encompassed by the term hematological malignancy. For example, hematological malignancies also include cancers of additional hematopoietic cells, including dendritic cells, platelets, erythrocytes, natural killer cells, and polymorphonuclear leukocytes, e.g., basophils, eosinophils, neutrophils and monocytes. It should be clear to those of skill in the art that these pre-malignancies and malignancies will often have different names due to changing systems of classification, and that patients having lymphomas classified under different names may also benefit from the therapeutic regimens of the present invention.

As mentioned, for this aspect of the present invention, the cancer does not include one associated with an Adenomatous polyposis coli (APC) mutation.

Examples of APC mutations are for instance those which cause truncation of the APC product. Typically mutations occur in the first half of the coding sequence, and somatic mutations in colorectal tumors are further clustered in a particular region, called MCR (mutation cluster region). A list of APC mutations involved in human disease are provided in OMIM, worldwidewebdotncbidotnlmdotnihdotgov/omim. Examples of cancers associated with APC mutations include colorectal cancer, medulloblastoma and hepatocellular carcinoma.

According to another aspect of the present invention there is provided a method of treating CML in a subject in need thereof, comprising administering to the subject a therapeutically effective amount of a Casein kinase I inhibitor, wherein the CML is selected from the group consisting of imatinib-resistant CML, imatinib (or imatinib-related TKI)-intolerant CML, accelerated CML, and lymphoid blast phase CML.

According to still another aspect of the present invention there is provided a method of treating a cancer in a subject in need thereof, comprising administering to the subject a therapeutically effective amount PF670462, wherein the cancer is not CLL.

All cancers are contemplated for this aspect of the invention (except for CLL). According to one embodiment of this aspect of the invention the cancer includes those associated with APC mutations as well. According to another embodiment, the cancer does not include those associated with APC mutations.

The methods of treating of the present invention are effected by contacting/administering an agent capable of inhibiting CKI.

CKI is a well-conserved family of Ser/Thr kinases found in every organism tested, from yeast to man. In mammals, the CKI family is composed of seven genes (α, β, γ₁, γ₂, γ₃, δ, ε) encoding 11 alternatively spliced isoforms. Members of the CKI family share a conserved catalytic domain and ATP-binding site, which exclusively differentiate them from other kinase families. CKI is a ubiquitous enzyme found in all cells, occupies different sub-cellular localizations and is involved in various cellular processes besides Wnt signaling.

Preferably, the CKI inhibitors increase the expression and/or activity of p53 (by at least 2 fold) and/or activate a DNA Damage Response (DDR).

CKI inhibitors of the invention preferably have at least twice, at least 5 times, at least 10 times the inhibitors activity towards CKI as compared to other kinases such as Cyclin Dependent Kinases (CDK) regulating cell cycle, (e.g. Cdk2, Cdk4, Cdk6). In addition, CKI inhibitors have at least twice, at least 5 times, at least 10 times the inhibitors activity towards CKI as compared to protein kinase C(PKC), PKA, her2, raf 1, MEK1, MAP kinase, EGF receptor, PDGF receptor, IGF receptor, PI3 kinase, weel kinase, Src, and/or Abl.

In some embodiments, the agents are CKI-alpha inhibitors i.e. they are selective towards CKI-alpha (CSNK1A; at the genomic, mRNA or protein level, GenBank Accession Nos. NP_001020276 and NM_001025105 and NM_001020276). Thus, for example, such CKI inhibitors have at least twice, at least 5 times, at least 10 times the inhibitors activity towards CKI-alpha as compared to CKI-delta and CKI-epsilon.

Preferably, the agents that are selective towards CKI-alpha are at least as effective as upregulating p53 as inhibitors of CKI delta and epsilon (e.g. PF670462). Preferably, the agents that are selective towards CKI-alpha are at least twice as effective as upregulating p53 as inhibitors of CKI delta and epsilon (e.g. PF670462).

In some embodiments the agents inhibit CKI-delta (CSNK1A; at the genomic, mRNA or protein level, GenBank Accession Nos. NP_001884.2, NP_620693.1, NM_001893.3 and NM_139062.1) and CKI-epsilon (CSNK1E; NP_001885.1, NP_689407.1, NM_001894.4 NM_152221.2).

In some embodiments of aspects of the present invention, the agents inhibit CKI-delta and CKI-epsilon to a greater extent than they inhibit CKI-alpha (e.g. at least twice, at least 5 times, at least 10 times the inhibitors activity towards CKI-delta and CKI-epsilon as compared to CKI-alpha.

In some embodiments of aspects of the present invention, the CKI inhibitors inhibit CKI alpha, delta and epsilon isoforms to a greater extent than they inhibit CKI-β, γ₁, γ₂, or γ₃ (e.g. at least twice, at least 5 times, at least 10 times the inhibitors activity towards CKI-delta and CKI-epsilon as compared to any of CKI β, γ₁, γ₂, or γ₃.

According to one embodiment, the CKI inhibitors of the present invention bind directly to the CKI (e.g. CKI-alpha, CKI-delta and/or CKI-epsilon) or a gene encoding same.

Downregulation of CKI-alpha, CKI-delta and/or CKI-epsilon can be effected on the genomic and/or the transcript level using a variety of molecules that interfere with transcription and/or translation (e.g., antisense, siRNA, Ribozyme, micro RNA or DNAzyme), or on the protein level using, e.g., antagonists, enzymes that cleave the polypeptide, and the like.

One example of an agent capable of downregulating the CKI's of the present invention is an antibody or antibody fragment capable of specifically binding the specific CKI. Preferably, the antibody specifically binds at least one epitope of CKI-alpha, CKI-delta or CKI-epsilon.

As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three-dimensional structural characteristics, as well as specific charge characteristics.

Methods for humanizing non-human antibodies are well known in the art. Generally, a humanized antibody has one or more amino acid residues from a non-human source introduced into it. These non-human amino acid residues are often referred to as import residues, which are typically taken from an import variable domain. Humanization can be essentially performed following the method of Winter and co-workers (see Jones et al. (1986); Riechmann et al. (1988); and Verhoeyen, M. et al. (1988). Reshaping human antibodies: grafting an antilysozyme activity. Science 239, 1534-1536), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom, H. R. and Winter, G. (1991). By-passing immunization. Human antibodies from synthetic repertoires of germline VH gene segments rearranged in vitro. J Mol Biol 227, 381-388). The techniques of Cole et al. and Boerner et al. are also available for the preparation of human monoclonal antibodies (Cole et al. (1985), Monoclonal Antibodies and Cancer Therapy, Alan R. Liss, Inc., pp. 77-96; and Boerner, P. et al. (1991). Production of antigen-specific human monoclonal antibodies from in vitro-primed human splenocytes. J Immunol 147, 86-95). Similarly, human antibodies can be made by introduction of human immunoglobulin loci into transgenic animals, e.g., mice, in which the endogenous immunoglobulin genes have been partially or completely inactivated. Upon challenge, human antibody production is observed to closely resemble that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016; and in the following scientific publications: Marks, J. D. et al. (1992). By-passing immunization: building high affinity human antibodies by chain shuffling. Biotechnology (N.Y.) 10(7), 779-783; Lonberg et al., 1994. Nature 368:856-859; Morrison, S. L. (1994). News and View: Success in Specification. Nature 368, 812-813; Fishwild, D. M. et al. (1996). High-avidity human IgG kappa monoclonal antibodies from a novel strain of minilocus transgenic mice. Nat Biotechnol 14, 845-851; Neuberger, M. (1996). Generating high-avidity human Mabs in mice. Nat Biotechnol 14, 826; and Lonberg, N. and Huszar, D. (1995). Human antibodies from transgenic mice. Int Rev Immunol 13, 65-93.

Another example of an agent capable of downregulating the CKIs of the present invention is an RNA silencing agent.

As used herein, the term “RNA silencing” refers to a group of regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

It will be appreciated that siRNA may be designed to inhibit more than one CKI (e.g. both CKI-delta and CKI-epsilon) by selecting sequences that are shared by both proteins. An exemplary siRNA capable of down-regulating CKI-alpha is as set forth in SEQ ID NOs: 1 and 2. An exemplary siRNA capable of down-regulating CKI-delta is as set forth in SEQ ID NO: 6 (5′-GAAACAUGGUGUCCGGUUUTT-3). An exemplary siRNA capable of down-regulating CKI-epsilon is as set forth in SEQ ID NO: 5. An exemplary siRNA capable of down-regulating both CKI-delta and CKI-epsilon is set forth in SEQ ID NOs: 3 and 4.

Silencer RNAs for the CKIs of the present invention are also commercially available—for example from Applied Biosystems.

The strands of a double-stranded interfering RNA (e.g., an siRNA) may be connected to form a hairpin or stem-loop structure (e.g., an shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

Synthesis of RNA silencing agents suitable for use with the present invention can be effected as follows. First, the CKI mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

Another agent capable of downregulating a CKI of the present invention is a DNAzyme molecule, which is capable of specifically cleaving an mRNA transcript or a DNA sequence of the CKI-alpha, delta or epsilon. DNAzymes are single-stranded polynucleotides that are capable of cleaving both single- and double-stranded target sequences (Breaker, R. R. and Joyce, G. F. (1995). A DNA enzyme with Mg²⁺-dependent RNA phosphoesterase activity. Curr Biol 2, 655-660; Santoro, S. W. and Joyce, G. F. (1997). A general purpose RNA-cleaving DNA enzyme. Proc Natl Acad Sci USA 94, 4262-4266). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro and Joyce (1997)); for review of DNAzymes, see: Khachigian, L. M. (2002). DNAzymes: cutting a path to a new class of therapeutics. Curr Opin Mol Ther 4, 119-121.

Examples of construction and amplification of synthetic, engineered DNAzymes recognizing single- and double-stranded target cleavage sites are disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh, T. et al., Abstract 409, American Society of Gene Therapy 5th Annual Meeting (www.asgt.org), Jun. 5-9, 2002, Boston, Mass. USA.). In another application, DNAzymes complementary to bcr-ab1 oncogenes were successful in inhibiting the oncogene's expression in leukemia cells, and in reducing relapse rates in autologous bone marrow transplants in cases of Chronic Myelogenous Leukemia (CML) and Acute Lymphoblastic Leukemia (ALL).

Downregulation of the CKI of the present invention can also be effected by using an antisense polynucleotide capable of specifically hybridizing with an mRNA transcript encoding the CKI.

Design of antisense molecules that can be used to efficiently downregulate a CKI must be effected while considering two aspects important to the antisense approach. The first aspect is delivery of the oligonucleotide into the cytoplasm of the appropriate cells, while the second aspect is design of an oligonucleotide that specifically binds the designated mRNA within cells in a manner inhibiting the translation thereof.

The prior art teaches of a number of delivery strategies which can be used to efficiently deliver oligonucleotides into a wide variety of cell types (see, for example: Luft, F. C. (1998). Making sense out of antisense oligodeoxynucleotide delivery: getting there is half the fun. J Mol Med 76(2), 75-76 (1998); Kronenwett et al. (1998). Oligodeoxyribonucleotide uptake in primary human hematopoietic cells is enhanced by cationic lipids and depends on the hematopoietic cell subset. Blood 91, 852-862; Rajur, S. B. et al. (1997). Covalent protein-oligonucleotide conjugates for efficient delivery of antisense molecules. Bioconjug Chem 8, 935-940; Lavigne et al. Biochem Biophys Res Commun 237: 566-71 (1997); and Aoki, M. et al. (1997). In vivo transfer efficiency of antisense oligonucleotides into the myocardium using HVJ-liposome method. Biochem Biophys Res Commun 231, 540-545).

In addition, also available are algorithms for identifying those sequences with the highest predicted binding affinity for their target mRNA based on a thermodynamic cycle that accounts for the energetics of structural alterations in both the target mRNA and the oligonucleotide (see, for example, Walton, S. P. et al. (1999). Prediction of antisense oligonucleotide binding affinity to a structured RNA target. Biotechnol Bioeng 65, 1-9).

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF-alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiencies of specific oligonucleotides using an in vitro system were also published (Matveeva, O. et al. (1998). Prediction of antisense oligonucleotide efficacy by in vitro methods. Nature Biotechnology 16, 1374-1375).

Several clinical trials have demonstrated the safety, feasibility, and activity of antisense oligonucleotides. For example, antisense oligonucleotides suitable for the treatment of cancer have been successfully utilized (Holmund, B. P. et al. (1999). Toward antisense oligonucleotide therapy for cancer: ISIS compounds in clinical development. Curr Opin Mol Ther 1, 372-385), while treatment of hematological malignancies via antisense oligonucleotides targeting c-myb gene, p53, and Bcl-2 entered clinical trials and was shown to be tolerated by patients (Gewirtz, A. M. (1999). Oligonucleotide therapeutics: clothing the emperor. Curr Opin Mol Ther 1, 297-306).

More recently, antisense-mediated suppression of human heparanase gene expression was reported to inhibit pleural dissemination of human cancer cells in a mouse model (Uno, F. et al. (2001). Antisense-mediated suppression of human heparanase gene expression inhibits pleural dissemination of human cancer cells. Cancer Res 61, 7855-7860).

Thus, the current consensus is that recent developments in the field of antisense technology, which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a CKI is a ribozyme molecule capable of specifically cleaving an mRNA transcript encoding the specific CKI. Ribozymes increasingly are being used for the sequence-specific inhibition of gene expression by the cleavage of mRNAs encoding proteins of interest (Welch, P. J. et al. (1998). Expression of ribozymes in gene transfer systems to modulate target RNA levels. Curr Opin Biotechnol 9, 486-496). The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers, and specific somatic mutations in genetic disorders (Welch, P. J. et al. (1998). Ribozyme gene therapy for hepatitis C virus infection. Clin Diagn Virol 10, 163-171). Most notably, several ribozyme gene therapy protocols for HIV patients are already in Phase 1 trials. More recently, ribozymes have been used for transgenic animal research, gene target validation, and pathway elucidation. Several ribozymes are in various stages of clinical trials. ANGIOZYME™ was the first chemically synthesized ribozyme to be studied in human clinical trials. ANGIOZYME specifically inhibits formation of the VEGFR (Vascular Endothelial Growth Factor receptor), a key component in the angiogenesis pathway. Ribozyme Pharmaceuticals, Inc., as well as other firms, has demonstrated the importance of anti-angiogenesis therapeutics in animal models. HEPTAZYME™, a ribozyme designed to selectively destroy Hepatitis C Virus (HCV) RNA, was found effective in decreasing Hepatitis C viral RNA in cell culture assays (Ribozyme Pharmaceuticals, Inc., Boulder, Colo., USA (www.rpi.com)).

An additional method of regulating the expression of a CKI gene in cells is via triplex-forming oligonucleotides (TFOs). Recent studies show that TFOs can be designed to recognize and bind to polypurine or polypirimidine regions in double-stranded helical DNA in a sequence-specific manner. These recognition rules are outlined in: Maher III, L. J., et al. (1989). Inhibition of DNA binding proteins by oligonucleotide-directed triple helix formation. Science 245, 725-730; Moser, H. E., et al. (1987). Sequence-specific cleavage of double helical DNA by triple helix formation. Science 238, 645-650; Beal, P. A. and Dervan, P. B. (1991). Second structural motif for recognition of DNA by oligonucleotide-directed triple-helix formation. Science 251, 1360-1363; Cooney, M., et al. (1988). Science 241, 456-459; and Hogan, M. E., et al., EP Publication 375408. Modifications of the oligonucleotides, such as the introduction of intercalators and backbone substitutions, and optimization of binding conditions (e.g., pH and cation concentration) have aided in overcoming inherent obstacles to TFO activity such as charge repulsion and instability, and it was recently shown that synthetic oligonucleotides can be targeted to specific sequences (for a recent review, see Seidman, M. M. and Glazer, P. M. (2003). The potential for gene repair via triple helix formation J Clin Invest 112, 487-494).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple-helical stability (Reither, S. and Jeltsch, A. (2002). Specificity of DNA triple helix formation analyzed by a FRET assay. BMC Biochem 3(1), 27, Epub). The same authors have demonstrated that TFOs designed according to the A-AT and G-GC rule do not form nonspecific triplexes, indicating that triplex formation is indeed sequence-specific.

Thus, a triplex-forming sequence may be devised for any given sequence in the CKI regulatory region. Triplex-forming oligonucleotides preferably are at least 15, more preferably 25, still more preferably 30 or more, nucleotides in length, up to 50 or 100 bp.

Transfection of cells with TFOs (for example, via cationic liposomes) and formation of the triple-helical structure with the target DNA induces steric and functional changes, blocking transcription initiation and elongation, allowing the introduction of desired sequence changes in the endogenous DNA, and resulting in the specific downregulation of gene expression. Examples of suppression of gene expression in cells treated with TFOs include: knockout of episomal supFG1 and endogenous HPRT genes in mammalian cells (Vasquez, K. M. et al. (1999). Chromosomal mutations induced by triplex-forming oligonucleotides in mammalian cells. Nucl Acids Res 27, 1176-1181; and Puri, N. et al. (2001). Targeted Gene Knockout by 2′-O-Aminoethyl Modified Triplex Forming Oligonucleotides. J Biol Chem 276, 28991-28998); the sequence- and target-specific downregulation of expression of the Ets2 transcription factor, important in prostate cancer etiology (Carbone, G. M. et al., Selective inhibition of transcription of the Ets2 gene in prostate cancer cells by a triplex-forming oligonucleotide. Nucl Acids Res 31, 833-843); and regulation of the pro-inflammatory ICAM-1 gene (Besch, R. et al. (2003). Specific inhibition of ICAM-1 expression mediated by gene targeting with Triplex-forming oligonucleotides. J Biol Chem 277, 32473-32479). In addition, Vuyisich and Beal have recently shown that sequence-specific TFOs can bind to dsRNA, inhibiting activity of dsRNA-dependent enzymes such as RNA-dependent kinases (Vuyisich, M. and Beal, P. A. (2000). Regulation of the RNA-dependent protein kinase by triple helix formation. Nucl Acids Res 28, 2369-2374).

Additionally, TFOs designed according to the abovementioned principles can induce directed mutagenesis capable of effecting DNA repair, thus providing both downregulation and upregulation of expression of endogenous genes (Seidman and Glazer (2003)). Detailed description of the design, synthesis, and administration of effective TFOs can be found in U.S. patent application Ser. Nos. 03/017,068 and 03/009,6980 to Froehler et al. and Ser. No. 02/012,8218 and 02/012,3476 to Emanuele et al., and U.S. Pat. No. 5,721,138 to Lawn.

MicroRNAs can be designed using the guidelines found in the art. Algorithms for design of such molecules are also available. See e.g., www.wmddotweigelworlddotorg/cgi-bin/mirnatoolsdotpl, herein incorporated by reference.

Another agent capable of downregulating the CKIs of the present invention is any molecule which binds to and/or cleaves the CKI. Such molecules can be, for instance, CKI antagonists, or a CKI inhibitory peptide.

It will be appreciated that a non-functional analogue of at least a catalytic or binding portion of CKI can be also used as an agent which downregulates CKI.

Small chemical CKI inhibitors are also contemplated by the present invention. These chemical agents may have selective inhibitory activities towards one particular CKI or may comprise inhibitory activities towards two or more CKIs. Such inhibitors may have at least two fold, at least five fold or even ten fold greater inhibitory activity towards CKI-delta and epsilon as compared with its inhibitory activity towards CKI-alpha For example, IC261 (available from Santa Cruz technology) is a specific inhibitor of the CKI-delta and CKI-epsilon.

According to a particular embodiment, the small chemical CKI inhibitor is selective towards CKI-delta. Such inhibitors may have at least two fold, at least five fold or even ten fold greater inhibitory activity towards CKI-delta as compared with its inhibitory activity towards CKI-alpha and/or CKI-epsilon.

According to another embodiment, the small chemical CKI inhibitor is selective towards CKI-epsilon. Such inhibitors may have at least two fold, at least five fold or even ten fold greater inhibitory activity towards CKI-epsilon as compared with its inhibitory activity towards CKI-alpha and/or CKI-delta.

According to another embodiment, the small molecule, chemical agent (i.e. not a polynucleotide agent) has at least two fold, at least five fold or even ten fold greater inhibitory activity towards CKI-alpha as compared with its inhibitory activity towards CKI-delta and CKI-epsilon. According to one embodiment, the small molecule agent is at least as effective in upregulating p53 as an inhibitor of CKI delta and epsilon. Preferably, the small molecule agent is at least twice as effective in upregulating p53 as an inhibitor of CKI delta and epsilon.

According to a particular embodiment, the agent is not CKI7, D4476, or IC261 since none of these agents stabilize beta catenin and p53, nor do they induce a DNA damage response.

Contemplated small molecule agents include PF670462 (CAS No: 950912-80-8) or PF 4800567 (CAS No: 1188296-52-7).

Another agent that can be used according to the present invention to downregulate CKI is a molecule which prevents CKI activation or substrate binding.

Other agents which may be used to regulate CKI-alpha, delta or epsilon can be found or refined (for enhanced selectivity, specificity) using screening methods which are well known in the art. Examples of such assays include biochemical assays (e.g., in-vitro kinase activity), cell biology assays (e.g. protein localization) and molecular assays (e.g., Northern, Western and Southern blotting).

Below is a description of various assays that may be used to screen small chemical agents for the ability to down-regulate one of the CKIs of the present invention.

Enzyme inhibition assays:
1. Incubate recombinant CKlepsilon enzyme with a small molecule inhibitor (SMI) for 10 minutes; add the substrate human Per2 and observe Ser662 phosphorylation by protein upshift on SDS-PAGE (Toh et al, Science 291:1040, 2001).
2. Incubate recombinant CKldelta enzyme with an SMI for 10 minutes; add the substrate mouse p53 and observe Thr18 phosphorylation by Western blotting using Novus Rabbit Anti-p53, phospho (Thr18) Polyclonal Antibody (NB100-92607).
3. Incubate human tumor cells with an SMI for 1-24 hours; harvest the cells and analyze them for beta-catenin phosphorylation on Ser45 with Invitrogen Rabbit Anti-beta-Catenin, phospho (Ser45) Polyclonal Antibody (44-208G) (a unique property of CKlalpha)

Biological Assays
1. Incubate human tumor cells with an SMI for 1-24 hours; harvest the cells and analyze them for DDR and p53 activation with antibodies to γH2A.X and p53 by immunohistochemistry or Western Blotting.
2. Incubate human primary tumor cells and tumor-associated fibroblasts with an SMI for 24 hours; remove the SMI and replacing the culture medium; analyze the cells for cellular senescence by Senescence-Associated β-galactosidase assay (SA-β-Gal).

Candidate agents may include, small chemical inhibitors, antibodies or various polynucleotide agents such as those described herein above. Following identification using the screening methods listed above, the agents may be tested as a candidate anti-cancer agent on cancerous cells or as a candidate for depleting hematopoietic stem cells. Confirmation of agent activity may be followed by synthesizing larger amount of the agent and preparation thereof in a pharmaceutical composition comprising same as detailed herein below.

As mentioned, the inhibitors of the present invention may also be used as a hemato-ablation agent for depleting bone marrow cells prior to a cell transplantation procedure. The hemato-ablation may be performed in conjunction with chemotherapy and/or irradiation, or in the absence of chemotherapy and/or irradiation.

Thus, according to another aspect of the present invention there is provided a method of transplanting cells into a subject in need thereof comprising:

(a) depleting immature blood cells from a blood or bone marrow of a subject by contacting the immature blood cells from a blood or bone marrow with an amount of a CKI inhibitor which up-regulates an amount and/or activity of p53 and kills the immature blood cells in the blood or bone marrow; and subsequently

(b) transplanting cells into the subject.

According to this aspect of the present invention, the subject is suffering from a disease for which cell transplantation is therapeutic.

Such diseases include but are not limited to a hematological disease, a cardiac disease, diabetes, neurodegenerative disease, a malignant disease, an immune disease and an autoimmune disease. The disease may be congenital or acquired.

According to an embodiment of this aspect of the present invention, the disease is a malignant disease. According to a specific embodiment, the malignant disease is a malignancy of hematopoietic or lymphoid tissues.

Diseases from which the subject may be suffering from include, but are not limited to, leukemia [e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, T-cell acute lymphocytic leukemia (T-ALL) and B-cell chronic lymphocytic leukemia (B-CLL)1, lymphoma [e.g., Hodgkin's lymphoma, non-Hodgkin's lymphoma, B cell, diffuse large B-cell lymphoma (DLBCL), B-cell chronic lymphocytic leukemia/lymphoma, Burkitt's lymphoma, T cell, cutaneous T cell, precursor T-cell leukemia/lymphoma, follicular lymphoma, mantle cell lymphoma, MALT lymphoma, histiocytic, lymphoblastic, thymic and Mycosis fungoides], diseases associated with transplantation of a graft (e.g. graft rejection, chronic graft rejection, subacute graft rejection, hyper-acute graft rejection, acute graft rejection and graft versus host disease), autoimmune diseases such as Type 1 diabetes, severe combined immunodeficiency syndromes (SCID), including adenosine deaminase (ADA), osteopetrosis, aplastic anemia, Gaucher's disease, thalassemia and other congenital or genetically-determined hematopoietic abnormalities.

The immature blood cells which are depleted according to this aspect of the present invention includes hematopoietic stem cells (HSCs), hematopoietic progenitor cells and cancer stem cells. The immature blood cells may be present in the bone marrow and/or the circulatory blood.

The term “hematopoietic stem cell” refers to multipotent stem cells that give rise to all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). When transplanted into lethally irradiated animals or humans, hematopoietic stem cells can repopulate the erythroid, neutrophil-macrophage, megakaryocyte and lymphoid hematopoietic cell pool.

As used herein, the term “hematopoietic stem and progenitor cell” or “HSPC” refers to a cell identified by the presence of the antigenic marker CD34 and the absence of lineage (lin) markers. HSPCs are therefore characterized as CD34⁺/Lin(−) cells, and populations of such cells. It is recognized that the population of cells comprising CD34+ and Lin(−) cells also includes hematopoietic progenitor cells, and so for the purposes of this application the term “HSPC” includes hematopoietic progenitor cells.

As used herein, the term depleting refers to eliminating at least 10%, at least 20%, at least 30%, at least 40%, at least 50%, at least 60%, at least 70%, at least 80%, at least 90%, or 100% of the bone marrow stem cells.

Thus, the CKI inhibitor may be provided in a myeloablative or a myeloreductive dose.

As used herein, “myeloablative”, refers to a treatment in which death, due to marrow failure, in a significant number of recipients, will occur if hematopoietic stem cell transplantation is not given.

As used herein, “non-myeloablative”, refers to a treatment which kills marrow cells but will not, in a significant number of recipients, lead to death from marrow failure.

As used herein, “myeloreductive”, refers to a treatment which causes cytopenia or anemia.

It will be appreciated that when the CKI inhibitor is provided in a myeloreductive dose, additional agents may be used to bring about a full myeloablation, Such agents include for example cytoreductive agent selected from one or more of alkylating agents (e.g., nitrogen mustards [such as mechloretamine], cyclophosphamide, melphalan and chlorambucil), alkyl sulphonates (e.g., busulphan), nitrosoureas (e.g., carmustine, lomustine, semustine and streptozocine), triazenes (e.g., dacarbazine), antimetabolites (e.g., folic acid analogs such as methotrexate), pyrimidine analogs (e.g. fluorouracil and cytarabine), purine analogs (e.g., fludarabine, idarubicin, cytosine arabinoside, mercaptopurine and thioguanine), vinca alkaloids (e.g., vinblastine, vincristine and vendesine), epipodophyllotoxins (e.g., etoposide and teniposide), antibiotics (e.g., dactinomycin, daunorubicin, doxorubicin, bleomycin, plicamycin and mitomycin), dibromomannitol, deoxyspergualine, dimethyl myleran and thiotepa.

Additional myeloreductive non-myeloablative agents are alkylating agents, e.g., cyclophosphamide, or fludarabine or similar substances, however, hematopoietic space creating antibodies or drugs, e.g., inhibitors of cell proliferation, e.g., DSG, or an anti-metabolite, e.g. brequinar, or an anti-T cell antibody, e.g., one or both of an anti-CD4 or anti-CD8 antibody can be used as a myeloreductive non-myeloablative agent. X-radiation and a combination of X-radiation and drug administration is also contemplated.

In some embodiments, bone marrow ablation is produced by administration of radioisotopes known to kill metastatic bone cells, for example, radioactive strontium, ¹³⁵Samarium, or ¹⁶⁶Holmium (Applebaum et al., 1992, Blood 80:1608-1613).

The CKI inhibitor is typically provided in an amount which is capable of increasing the amount and/or activity of p53.

Contacting of the CKI inhibitor with the bone marrow cells may be effected in vivo or ex vivo—for example during apheresis.

Thus, according to another aspect of the present invention there is provided method of depleting immature blood cells from a blood or bone marrow of a subject comprising contacting the stem cells ex vivo with an amount of a CKI inhibitor which up-regulates an amount and/or activity of p53 and kills the immature blood cells in the blood or bone marrow, thereby depleting the immature blood cells from the blood or bone marrow.

CKI inhibitors which may be used according to these aspects of the present invention are described herein above.

The cells which are transplanted may be isolated cells (also referred to as a cell graft) or may be comprised in a tissue (also referred to as a tissue graft).

As used herein, the phrase “cell or tissue graft” refers to a bodily cell (e.g. a single cell or a group of cells) or tissue (e.g. solid tissues or soft tissues, which may be transplanted in full or in part). Exemplary tissues which may be transplanted according to the present teachings include, but are not limited to, lymphoid/hematopoietic tissues (e.g. lymph node, Peyer's patches thymus or bone marrow). Exemplary cells which may be transplanted according to the present teachings include, but are not limited to, hematopoietic stem cells (e.g. immature hematopoietic cells). According to a specific embodiment, the hematopoietic stem cells of the present invention are CD34+.

It will be appreciated that the type of cells which are transplanted into the subject following the bone marrow stem cell depletion is dependent on the disease being treated.

Thus, for example, when the subject has renal or heart failure, the transplanted cells may comprise kidney or cardiac cells. When the subject is suffering from hepatic or lung failure or skin damage (e.g., burns), the graft may comprise liver, lung or skin tissue. When the subject has diabetes, the cells comprise beta cell pancreatic cells. When the subject has a hematological disease, the cells may comprise immature hematopoietic cells.

Depending on the application, the method may be effected using a cell or tissue graft which is syngeneic or non-syngeneic with the subject.

As used herein, the term “syngeneic” refers to a cell or tissue which is derived from an individual who is essentially genetically identical with the subject. Typically, essentially fully inbred mammals, mammalian clones, or homozygotic twin mammals are syngeneic.

Examples of syngeneic cells or tissues include cells or tissues derived from the subject (also referred to in the art as “autologous”), a clone of the subject, or a homozygotic twin of the subject.

As used herein, the term “non-syngeneic” refers to a cell or tissue which is derived from an individual who is allogeneic or xenogeneic with the subject's lymphocytes (also referred to in the art as “non-autologous”).

As used herein, the term “allogeneic” refers to a cell or tissue which is derived from a donor who is of the same species as the subject, but which is substantially non-clonal with the subject. Typically, outbred, non-zygotic twin mammals of the same species are allogeneic with each other. It will be appreciated that an allogeneic donor may be HLA identical or HLA non-identical with respect to the subject.

As used herein, the term “xenogeneic” refers to a cell or tissue which substantially expresses antigens of a different species relative to the species of a substantial proportion of the lymphocytes of the subject. Typically, outbred mammals of different species are xenogeneic with each other.

The present invention envisages that xenogeneic cells or tissues are derived from a variety of species such as, but not limited to, bovines (e.g., cow), equids (e.g., horse), porcines (e.g. pig), ovids (e.g., goat, sheep), felines (e.g., Felis domestica), canines (e.g., Canis domestica), rodents (e.g., mouse, rat, rabbit, guinea pig, gerbil, hamster) or primates (e.g., chimpanzee, rhesus monkey, macaque monkey, marmoset).

Cells or tissues of xenogeneic origin (e.g. porcine origin) are preferably obtained from a source which is known to be free of zoonoses, such as porcine endogenous retroviruses. Similarly, human-derived cells or tissues are preferably obtained from substantially pathogen-free sources.

According to an embodiment of the present invention, both the subject and the donor are humans.

Depending on the application and available sources, the cells or tissue grafts of the present invention may be obtained from a prenatal organism, postnatal organism, an adult or a cadaver donor. Moreover, depending on the application needed, the cells or tissues may be naive or genetically modified. Such determinations are well within the ability of one of ordinary skill in the art.

Any method known in the art may be employed to obtain a cell or tissue (e.g. for transplantation).

According to a particular embodiment, the cells which are transplanted comprise hematopoietic cells—e.g. immature hematopoietic cells.

As used herein, the term “immature hematopoietic cells” refers to any type of incompletely differentiated cells which are capable of differentiating into one or more types of fully differentiated hematopoietic cells. Immature hematopoietic cells include without limitation types of cells referred to in the art as “progenitor cells”, “precursor cells”, “stem cells”, “pluripotent cells”, “multipotent cells”, and the like.

Preferably the immature hematopoietic cells are hematopoietic stem cells.

Preferably, where the immature hematopoietic cells are derived from a human, the immature hematopoietic cells are CD34+ cells, such as CD34+CD133+ cells.

Types of grafts of the present invention which comprise immature hematopoietic cells include whole bone marrow cell grafts (T-cell depleted or non-T-cell-depleted), grafts of immature hematopoietic cells from bone marrow aspirates, grafts of peripheral blood-derived immature hematopoietic cells and grafts of umbilical cord-derived immature hematopoietic cells. Methods of obtaining such grafts are described hereinbelow.

A graft which comprises human peripheral blood-derived hematopoietic stem cells may be obtained according to standard methods, for example by mobilizing CD34+ cells into the peripheral blood by cytokine treatment of the donor, and harvesting of the mobilized CD34+ cells via leukapheresis. Ample guidance is provided in the literature of the art for practicing isolation of bone marrow-derived stem cells from the bone marrow or the blood (refer, for example, to: Arai S, Klingemann H G., 2003. Arch Med Res. 34:545-53; and Repka T. and Weisdorf D., 1998. Curr Opin Oncol. 10:112-7; Janssen W E. et al., 1994. Cancer Control 1:225-230; Atkinson K., 1999. Curr Top Pathol. 92:107-36).

A graft of human umbilical cord blood-derived hematopoietic stem cells may be obtained according to standard methods (refer, for example, to: Quillen K, Berkman E M., 1996. J. Hematother. 5:153-5).

A graft of hematopoietic stem cells of the present invention may also be derived from liver tissue or yolk sac.

A requisite number of hematopoietic stem cells can be provided by ex-vivo expansion of primary hematopoietic stem cells (reviewed in Emerson, 1996, Blood 87:3082, and described in more detail in Petzer et al., 1996, Proc. Natl. Acad. Sci. U.S.A. 3:1470; Zundstra et al., 1994, BioTechnology 12:909; and WO 95 11692).

Transplanting the cell or tissue graft into the subject may be effected in numerous ways, depending on various parameters, such as, for example, the cell or tissue type; the type, stage or severity of the recipient's disease (e.g. organ failure); the physical or physiological parameters specific to the subject; and/or the desired therapeutic outcome.

Transplanting a cell or tissue graft of the present invention may be effected by transplanting the cell or tissue graft into any one of various anatomical locations, depending on the application. The cell or tissue graft may be transplanted into a homotopic anatomical location (a normal anatomical location for the transplant), or into an ectopic anatomical location (an abnormal anatomical location for the transplant). Depending on the application, the cell or tissue graft may be advantageously implanted under the renal capsule, or into the kidney, the testicular fat, the sub cutis, the omentum, the portal vein, the liver, the spleen, the bones, the heart cavity, the heart, the chest cavity, the lung, the skin, the pancreas and/or the intra abdominal space.

It will be appreciated that the syngeneic or non-syngeneic hematopoietic cells (e.g. immature hematopoietic cells) of the present invention may be transplanted into a recipient using any method known in the art for cell transplantation, such as but not limited to, cell infusion (e.g. I.V.) or via an intraperitoneal route.

Optionally, when transplanting a cell or tissue graft of the present invention into a subject having a defective organ/cells, it may be advantageous to first at least partially remove the failed organ/cells from the subject so as to enable optimal development of the graft, and structural/functional integration thereof with the anatomy/physiology of the subject.

Prior to the depleting step, mobilization of the immature blood cells from the bone marrow to the blood is also contemplated by the present invention. Examples of mobilizing agents include growth factors or cytokines that affect mobilization, for example colony stimulating factors (e.g. granulocyte-colony stimulating factor, G-CSF and granulocyte-macrophages colony stimulating factor, GM-CSF) and stem cell factor, SCF. Peptide mobilization agents are also contemplated by the present invention including those disclosed in U.S. Patent Application Publication No. 2004/0209921, U.S. Pat. Nos. 6,946,445, 6,875,738, U.S. Patent Application Publication No. 2005/0002939, WO 2002/020561, WO 2004/020462 and WO 2004/087068, WO 00/09152, US 2002/0156034, and WO 2004/024178 and WO 01/85196.

Following transplantation of the cell or tissue graft into the subject according to the present teachings, it is advisable, according to standard medical practice, to monitor the growth functionality and immuno-compatability of the organ/cells according to any one of various standard art techniques. For example, structural development of the cells or tissues may be monitored via computerized tomography or ultrasound imaging while engraftment of non-syngeneic cell or bone marrow grafts can be monitored for example by chimerism testing [e.g. by PCR-based procedures using short tandem repeat (STR) analysis].

The CKI inhibitors described hereinabove (or expression vectors encoding polynucleotide CKI inhibitors) may be administered to the individual per se or as part of a pharmaceutical composition, which also includes a physiologically acceptable carrier. The purpose of a pharmaceutical composition is to facilitate administration of the active ingredient to an organism.

As used herein a “pharmaceutical composition” refers to a preparation of one or more (e.g. a CKI-alpha inhibitor, CKI-delta inhibitor and/or a CKI-epsilon inhibitor) of the active ingredients described herein with other chemical components such as physiologically suitable carriers and excipients.

Herein the term “active ingredient” refers to the agent (e.g., silencing molecule) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of an aggregate of cells having a similar structure and/or a common function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue. In an exemplary embodiment the tissue is a colon cancer tissue.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models (e.g., the APC model exemplified herein) to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The kit may comprise a combination of the inhibitors, such as a CKI-alpha inhibitor, CKI-delta inhibitor and a CKI-epsilon inhibitor. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

According to a particular embodiment, the subject is not concomitantly treated with Imatinib, Dastinib or Nilotinib.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Conditional CKIα KO Mice:

C57bl/6 mice with loxP flanked CSNK1A1 mice (Elyada et al., 2011) were crossed with mx1-Cre mice (Kuhn et al., 1995). Seven generations were backcrossed with C57bl/6 mice to generate a pure genetic background. Mx1-Cre induction was performed by three I.P. injection of 10 uL/g mouse of a 2 mg/mL Polyinosinic-polycytidylic acid sodium salt (pIpC) (sigma P1530) every other day. Engraftment was performed by I.V. injection of freshly isolated 5×10⁶ bone marrow cells.

BCR-ABL-Inducible CML Model:

To generate the BCR-ABL-inducible CML model, BM cells from MxCre⁻ Ck1α1^{fl/fl} or MxCre⁺ Ck1 α1^{fl/fl} were extracted and enriched for cKit expressing cells (EasySep #18757) and incubated overnight in RPMI supplemented with 15% FCS L-Glutamine, Pen/Strep (Beit Haemek) and stem cell factor (SCF), IL-3, IL-6 and TPO (Peprotech). The culture was then infected with p210BCR-ABL-IRES-GFP retrovirus construct containing supernatant medium for 4 hours and returned to medium for additional 24 h. The culture was then injected I.V. into sub-lethally irradiated (500 rad) mice. Upon detectable steady increase of GFP expressing cells in the mice peripheral blood (by FACS) and rise in leukocyte numbers and immature cells (detected by Wright-Giemsa stained blood films) the mice were sacrificed and their bone marrow was transferred to sub-lethally irradiated WT hosts.

Each such transfer was termed disease generation. By the fourth transfer, the hosts were no longer sub-lethally irradiated prior to disease transfer and the time between generations was shorter (usually 10 days). Blast crisis development was easily detectable by the highly abnormal number of blast cells (more than 30% of WBC in PB) and the shortening time between transfers. The experimental procedure is illustrated in FIG. 3. Experiments were performed on late generation diseases in which blasts were easily detectable, no irradiation of hosts was necessary and the generation time was short (up to 14 days). Mice were monitored daily for cachexia, lethargy, and ruff coats, paralysis and moribund mice were sacrificed.

For evaluating Ck1α 1 KO effect on CML, pIpC was administered by I.P. (20 μg/g mouse) every other day starting from 24 h after bone marrow transplantation (BMT).

The same procedure was performed when MxCre⁺Ck1α 1^{fl/fl} CD45.2 disease hosts were used, with the addition of I.V. 5×10⁶ BM cells from a WT congenic CD45.1 donor at days 7 from first pIpC administration. Chimerism was evaluated through analysis of CD45.1 expressing leukocytes in mice PB one month following engraftment. LT-HSC engraftment was assessed by the appearance of both myeloid and lymphoid mature CD45.1 expressing leukocytes in the PB.

The PF670462 was dissolved in 20% 2-hydroxypropyl β-cyclodextrin (vehicle) and administered by daily I.P. of 60 mg/Kg starting 7 hour after disease transfer. The control mice were treated with the vehicle only.

In Vitro Inhibitor Tests:

Freshly isolated bone marrow from CML carrying mice was mixed with normal mice bone marrow in a 1:1 ratio and grown in RPMI supplemented with 15% FCS L-Glutamine, Pen/Strep, Hepes, Sodium Pyruvate, non-essential amino acids (Beit Haemek). PF670462 inhibitor was dissolved in DMSO and added to the tissue culture medium at the indicated concentrations and 0.1% DMSO. As for control, the cells were treated with vehicle only. After 36-48 h, cells were harvested and counted manually using a camera and standard inverted light microscope. Dead cells were excluded using Trypan Blue (sigma). The number of normal and BCR-ABL expressing cells was later extrapolated according to FACS analysis of % GFP⁺/7AAD⁻ expressing cells. AnnexinV-PE (MBL), 7AAD (Tonbo) staining was evaluated by FACS according to manufacturer's recommendation.

Quantitative RT-PCR:

Total RNA from cells was extracted using DirectZol RNA miniprep (Zymed). cDNA was generated using a poly(dT) oligonucleotides (IDT) and MMLV—Reverse Transcriptase (Invitrogen) and amplified on a 7900HT Real Time PCR System (Applied Biosystems) using Platinum® SYBR® Green (Invitrogen) according to the manufacturer's instructions. At least triplicate reactions were performed for each gene. Melting curve analysis was performed after each run to control for the nonspecific PCR products and primer dimers. Normalization was performed using PP1A, UBC and HPRT as an internal control.

Western Blot Analysis:

Whole cell lysate were extracted in the presence of protease and phosphatase inhibitors from the bone marrow of CML carrying mice treated with either vehicle or PF670462.

Protein extracts, separated by SUS-PAGE and transferred onto nitrocellulose membranes, were probed with antibodies against beta-catenin, c-Myc, p53 and HSP90. Proteins of interest were detected with HRP-conjugated Donkey/Rabbit anti-mouse IgG antibody (1:5000. GE Healthcare, Uppsala, Sweden) and visualized with the Pierce ECL Western blotting substrate (Thermo Scientific, Rockford, Ill.), according to the provided protocol.

FACS Analysis:

All assays were performed on BD's: FACS caliber, FACS ARIA sorter or LSR II machines. For staining cells were suspended in a 1% BSA/PBS buffer with 5 uM EDTA. Cells were then incubated with the appropriate antibody for 30 minutes on ice, washed and incubated with the proper secondary antibody according to the manufactures recommendations. Monoclonal antibodies specific for CD16 and CD32 (Miltenyi Biotec) were used for blockade of Fc receptors before staining. The antibodies used for cell surface labeling are listed in Table 1 herein below.

TABLE 1

| Name | Company | Catalogue |
|---|---|---|
| Lineage cocktail- Biotin | Miltenyibiotec | 130-092-613 |
| (CD5, CD45R (B220), | | |
| CD11b, Gr-1 (Ly-6G/C), 7-4, | | |
| and Ter-119) | | |
| c-Kit APC-eFluor780 | eBioscience | 47-1171 |
| Sca-1 PE-Cy7 | eBioscience | 25-5981 |
| Strepavidin-percp/cy5.5 | eBioscience | 45-4317-82 |

### Example 1

### CKIα Deletion Leads to Normal HSC Depletion Allowing Bone Marrow Reconstitution

Ck1α 1^{fl/fl} Mx-Cre transgenic mice were generated in order to analyze the effect of CKIα deletion in bone marrow. Mx-Cre is induced not only in the BM but also in the liver and spleen. To ensure that the phenotype observed is specific to CKIα deletion in the BM and not in other tissues, the bone marrow of Ck1α 1^{fl/fl} Mx-Cre transgenic with GFP mouse was injected into a lethally IR WT mouse. By doing that, it was ensured that upon pIpC injection to the recipient mouse CKIα deletion is effected only in the BM and not in other tissues. Long term (LT) engraftment was validated by determining stable donor GFP positive cells in the peripheral blood 2 months following the transplantation. Only upon validation of successful engraftment, was pIpC injected.

Upon CKIα KO induction (with pIpC) the mice develop a lethal pancytopenia due to reduced HSC numbers resulting in a 20 days median survival (FIG. 1C). However, if the pIpC-treated, BM CKIα-deleted mice were transplanted with WT bone marrow at day 7 from pIpC treatment, they were rescued and displayed high levels of chimerism (FIGS. 1D-F). This was maintained for over 3 months and includes both myeloid and lymphoid lineages (not shown) indicating a long-term bone marrow reconstitution. The control mice were unaffected by the pIpC induction shots and did not display over 1% chimerism upon engraftment (data not shown).

### Example 2

### CKIα Deleted Bone Marrow Prevents Bcr-Abl Driven Leukemia Genesis

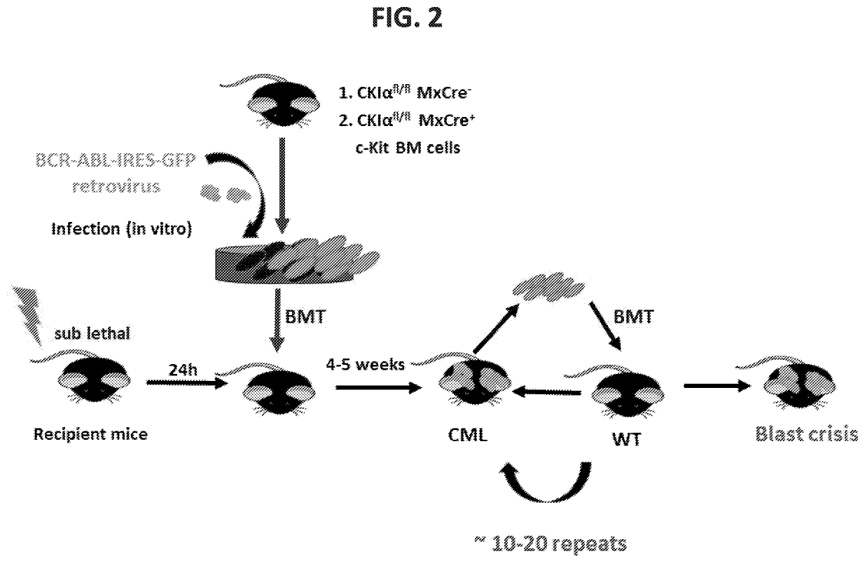

Bone marrow from mice carrying floxed alleles of CKIα with Mx-Cre or without were infected with a Bcr-Abl carrying retrovirus and injected into sub-lethally irradiated WT recipient mouse (FIG. 2). Next, the BM from the sick mouse was taken and engrafted into a WT mouse. This procedure was repeated multiple times until the chronic leukemia disease turned into an aggressive acute blast crisis disease, with multiple blast cell in the BM and peripheral blood and death within 2-3 weeks.

While in the first generation, the mice died after approximately 5 weeks, in the next generation the mice survived only two weeks after the transplantation because they suffer from a more aggressive disease. Furthermore, there was no further need to irradiate the leukemia recipient mouse after a few repeated transplantation, attesting to the aggressive nature of the leukemia.

To test the effect of CKIα ablation on CML development, pIpC was injected 24 h following leukemic BM engraftment to recipient mice. In this experiment, two different control groups were used: in the first control group the mice were injected with leukemia cells carrying the Mx-Cre and the mice were injected with PBS and in the second control group, the mice were injected with leukemia cell that lack the Mx-Cre but received pIpC injection (FIG. 3A-D). Disease progression was followed by counting the GFP+ in the peripheral blood of the recipient mice (FIG. 3B).

In the control mice, the GFP+ cells increased exponentially which indicate an aggressive disease while in the CKIα KO group the leukemic cells were almost undetectable (FIG. 3B). The difference between control and CKIα-deleted groups was also evident in blood smears. In the control group, multiple blasts were evident, as in CML blast crisis, while in the CKIα KO group the peripheral blood smear looked normal without blast cells (FIG. 3C). The mice were also followed for survival. While both control group mice died approximately 3 weeks following the bone marrow transfer, the vast majority of the CKIα KO group survived (FIG. 3D).

CML patients who are candidates for BMT are treated with high dose of chemotherapy or IR in order to eliminate the leukemia stem cells (LSC) prior to BMT. In these processes, the normal hematopoietic stem cells (HSCs) are eliminated as well.

To evaluate if CKIα deletion can substitute chemotherapy or irradiation-induces myeloablation and leukemia cell clearance, leukemic cells were injected into CKIα floxed with Mx-Cre mice.

In this model pIpC injection induced CKIα KO both in the leukemic and normal HSCs of the recipient mice. Seven days following KO induction, BM from WT mice was transplanted into the leukemic mice without irradiation (FIG. 4A). In order to differentiate between the donor and recipient mice, mice that harbor two different subtype of CD45 were used.

As illustrated in FIG. 4B, in the control group in which CKIα KO was not induced, no donor derived cell are evident in the peripheral blood (PB) while in the KO group more than 25% of the cells are donor derived following 12 days.

Analysis of disease progression showed that there was a very high percentage of leukemic cells in the control mouse while in the CKIα KO, the leukemic cells were undetectable (FIG. 4B), as mirrored by the survival rate data (FIG. 4C).

### Example 3

### Effect of the CKI Inhibitor PF670462 on Leukemia Cells

Next, the present inventors evaluated the effect of PF670462, a CKI inhibitor on normal BM and leukemia cell in vitro. PF670462 is considered CKI-delta/epsilon specific inhibitor (Long A, Zhao H, Huang X. J Med Chem. 2012 Jan. 26; 55(2):956-60. doi: 10.1021/jm201387s) and therefore is not supposed to activate the Wnt or p53 pathway (Price M A, Genes Dev. February 15; 20(4):399-410). When the cells were treated with the inhibitor in vitro, an upregulation of Wnt and p53 in a dose dependent manner was observed in bone marrow cell analysis (FIG. 5B), indicating CKIα inhibitory activity (Elyada et al, Nature. 2011 Feb. 17; 470(7334):409-13. doi: 10.1038/nature09673). Remarkably, a significant difference between the cell type after treatment with the inhibitor was observed, while the normal BM cell number was only slightly reduced upon treatment with increasing concentration of the inhibitor, the decline in the leukemic cell number was much more prominent (FIG. 5C).

It may be speculated that the inhibitor induced apoptosis in the leukemic cells. This was confirmed by the 7AAD−/Annexin+ assay which demonstrated an increase in apoptotic cell rate under the inhibitor treatment in a dosage dependent manner, while the WT BM cells are not significantly effected (FIG. 5D).

An in vivo study was performed as described in the scheme of FIG. 6A. Seven hours following disease transfer, mice were treating treated daily with PF670462 (i.p.). Western blot analysis of bone marrow cells harvested from the inhibitor-treated mice showed stabilization of β-catenin and p53 and induction of the Wnt target gene c-Myc, again attesting to CKIα inhibitory activity of PF670462 (FIG. 6B).

Disease progression in inhibitor-treated mice was followed by counting the GFP+ in the peripheral blood of the leukemia initiating cells (LIC) (i.e, cancer stem cells) recipient mice. In the group of mice that were treated with vehicle, the GFP+ cells went up exponentially which indicates an aggressive disease, while in the group of mice that were treated with CKI inhibitor the leukemic cells were almost undetectable (FIG. 6C). Whereas in vehicle-treated mice the bone marrow was invaded by leukemia cell destroying the vertebrates, inhibitor-treated mice had normal bone marrow appearance with intact vertebrates (FIG. 6D). Unlike vehicle-treated mice, no blast were detected in the peripheral blood of inhibitor-treated mice (FIG. 6E).

When the survival of the mice was followed, it can be seen that while the vehicle treated group died within 12 days, the CKI inhibitor treated group remained alive (FIG. 6F). Healthy-appearing inhibitor-treated mice were sacrificed after 20 days, their tissues examined for any signs of leukemia (e.g., FIG. 6D) and their bone marrow transplanted to lethally irradiated mice to determine if any residual LICs survived and expanded in the irradiated host and whether normal, long term repopulating hematopoietic stem cells (LT0HSCs) were affected by the inhibitor. So far, one month after transplantation, the recipient mouse bone marrow is fully reconstitute with no evidence of leukemia GFP+ cells in the peripheral blood, indicating intact LT-HSCs with complete elimination of LICs.

### Example 4

### Effect of CKIα Deletion in Melanoma Mice

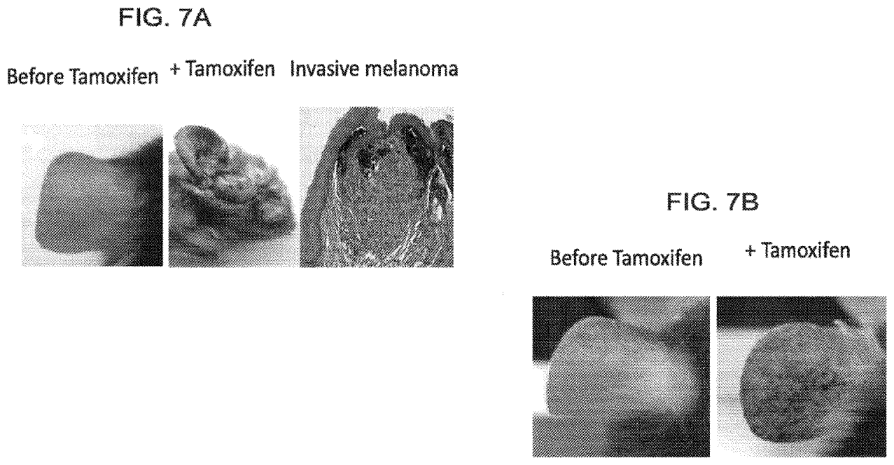

Mutational activation of BRAF is the earliest and most common genetic alteration in human melanoma. The expression of BRAfv600E combined with Pten tumor suppressor gene silencing elicits development of melanoma with 100% penetrance, short latency and with metastases observed in lymph nodes, peritoneal cavity and lungs. These mice provide a system to study melanoma's cardinal feature of metastasis with the presence of long-living melanoma initiating cells (MIC). The mouse melanoma model based on oncogenic BRAF and PTEN deletion (B6.Cg-Braf^{tm1Mmcm} Pten^{tm1Hwu} Tg(Tyr-cre/ERT2)13Bos/BosJ), based on tamoxifen-inducible activation of Tyrosinase-Cre (specific to melanocytes), referred to herein, the BRAF model was bred into the CKIα-floxed mice, referred to herein as the BRAF-CKI KO mouse model. Both the BRAF and the BRAF-CKI KO models were treated by topical ear application of tamoxifen. 56 days following tamoxifen treatment, the BRAF model mice developed metastatic melanoma (FIG. 7A), spreading locally and systemically, but the BRAF-CKI KO model showed no signs of melanoma, only pigmented spots on the ear (FIG. 7B). CKIα ablation therefore eliminates MICs in this experimental system.

BRAF melanoma mice are treated by daily 60 mg/Kg PF-670462 subcutaneously injections, or by the vehicle (20% 2-hydroxypropyl β-cyclodextrin; Sigma), beginning 24 hours or 3 weeks following tamoxifen induction of melanoma. Mice are sacrificed 56 days following tamoxifen induction so as to observe the effect of the inhibitor.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-4. (canceled)

5. A method of treating chronic myelogenous leukemia (CIVIL) in a subject in need thereof, comprising administering to the subject a therapeutically effective amount of a Casein kinase I inhibitor, wherein the CIVIL is selected from the group consisting of imatinib-resistant CML, imatinib-related TKI-resistant CML, imatinib-intolerant CML, accelerated CIVIL, and lymphoid blast phase CML, thereby treating the CIVIL.

6. (canceled)

7. A method of transplanting cells into a subject in need thereof comprising:
(a) depleting immature blood cells from a blood or bone marrow of a subject by contacting said immature blood cells from a blood or bone marrow with an amount of a CKI inhibitor which up-regulates an amount and/or activity of p53 and kills said immature blood cells in the blood or bone marrow; and subsequently:
(b) transplanting cells into the subject.

8. A method of depleting immature blood cells from a blood or bone marrow of a subject, comprising contacting the blood or bone marrow ex vivo with an amount of a CKI inhibitor which up-regulates the amount and/or activity of p53 and kills said immature blood cells in the blood or bone marrow, thereby depleting the immature blood cells from the blood or bone marrow.

9. (canceled)

10. The method of claim 7, further comprising inducing mobilization of said immature blood cells from the bone marrow to the blood prior to the depleting.

11. (canceled)

12. The method of claim 7, wherein said inhibitor binds to a CKIα or a polynucleotide encoding same.

13. (canceled)

14. The method of claim 7, wherein said inhibitor activates a DNA damage response (DDR).

15. The method of claim 7, wherein said CKI inhibitor comprises a CKIα inhibitory activity.

16. The method of claim 15, wherein said CKI inhibitor further comprises a CKI delta and/or CKI-epsilon inhibitory activity.

17. The method of claim 7, wherein said CKI inhibitor comprises a CKI delta and CKI-epsilon inhibitory activity.

18. The method of claim 7, wherein said inhibitor is a small molecule inhibitor.

19. The method of claim 7, wherein said inhibitor is PF670462.

20. The method of claim 7, wherein said inhibitor is an RNA silencing agent.

21. The method of claim 20, wherein said silencing agent is targeted against a CKIα.

22. The method of claim 7, wherein said immature blood cells comprise stem cells.

23. The method of claim 7, wherein said immature blood cells comprise cancer stem cells.

24. The method of claim 7, wherein said contacting is effected in vivo.

25. The method of claim 7, wherein said contacting is effected ex vivo.

26. The method of claim 25, wherein said contacting is effected during apheresis.

27. The method of claim 7, wherein said depleting is effected without irradiation or chemotherapy.

28. The method of claim 7, wherein said depleting is effected in combination with irradiation and/or chemotherapy.

29-41. (canceled)
